SEQUENCE LISTING (6 sequences)

SEQ ID NO: 1 - DNA, 22 bp, Human
actgggtagccttcaatgtgga

SEQ ID NO: 2 - DNA, 21 bp, Human
gacgagggcgtctctgagtag

SEQ ID NO: 3 - DNA, 22 bp, Human
cataaagaaagctcgcaacacg

SEQ ID NO: 4 - DNA, 22 bp, Human
actgctgaggaatgagatccag

SEQ ID NO: 5 - DNA, 20 bp, Human
cagcaccttctcgtgcatca

SEQ ID NO: 6 - DNA, 22 bp, Human
actgcacacagccaaagtagga

(19) United States
(12) Patent Application Publication
(10) Pub. No.: US 2010/0048514 A1
(43) Pub. Date: Feb. 25, 2010

(54) USE OF PHOSPHOLIPIDS FOR WOUND HEALING

(76) Inventors: Nick Topley, Cardiff (GB); Derek Woodcock, Herts (GB)

Correspondence Address: QUARLES & BRADY LLP, 411 E. WISCONSIN AVENUE, SUITE 2040, MILWAUKEE, WI, 53202-4497, US

(21) Appl. No.: 11/720,145

(22) PCT Filed: Nov. 28, 2005
(86) PCT No.: PCT/GB2005/004552

§ 371 Date: Nov. 10, 2009

(30) Foreign Application Priority Data
Nov. 26, 2004 (GB) 0426010.5

(51) Int. Cl. A61K 31/661; A61P 17/02

(52) U.S. Cl. 514/114; 514/121

(57) ABSTRACT

The present invention provides a phospholipid and a pharmaceutical composition comprising a phospholipid for use in the treatment of a wound by inducing hyaluronic acid secretion; and a method of treating a wound comprising the application of a phospholipid thereto.

The present invention provides a pharmaceutical composition for use in wound healing and a method for treatment of wounds. The composition comprises a phospholipid, particularly a mixture of phospholipids known as pumactant.

There is a general need to find ways of improving the healing rate for wounds. Tissue repair in a mammalian foetus is fundamentally different from normal healing. In an adult human, injured tissue is repaired by collagen deposition, collagen re-modelling and eventual scar formation, whereas foetal wound healing appears to be more of a regenerative process with minimal or no scar formation. An adult wound heals by the replacement of normal dermis with a scar that consists of excessive and abnormally organized collagen. In marked contrast, a foetal wound contains a persistent abundance of hyaluronic acid (otherwise known as hyaluronan) while collagen deposition is rapid and non-excessive, Bruce A Mast, M. D., Robert F. Diegelmann, Ph.D., Healing in the Mammalian Foetus, Surg. Gyn. And Ob., Vol. 174, pp. 441-451, May 1992.

What is known is that hyaluronic acid has a definite role in harnessing and manipulating the natural reparative capacity of tissue fibroblasts and the hyaluronic acid protein complexes play a significant role in vivo organization or scar tissue, D. A. R. Burd, R. M. Greco, S. Regaurer, M. T. Longaker, J. W. Siebert and H. G. Garg, Hyaluronan and Wound Healing: a New Perspective, Journal of Plastic Surgery, 1991, pp. 579-584.

It is understood that the term hyaluronic acid includes its derivatives and broadly refers to naturally occurring, microbial and synthetic derivatives of acidic polysaccharides of various molecular weights constituted by residues of glucuronic acid and N-acetyl-D-glucosamine.

The wound healing process is significantly different in adults as compared to the healing that takes place in amniotic fluid. Sharply increased levels of hyaluronic acid characterize adult wound healing during the first three days. By the seventh day, hyaluronic acid is not detectable.

Foetal wound healing is characterized by sharply increased levels of hyaluronic acid during the first three days, but unlike adult wound healing the level of hyaluronic acid remains elevated for 21 days. These findings are the result of research conducted by Michael T. Longaker, M.D., Ernie R. Harrison, M.D., and Robert Stern, M.D. and reported in an article titled Studies in Foetal Wound Healing: V. A. Prolonged Presence of Hyaluronic Acid Characterizes Foetal Wound Fluid, in Ann. Surg., April 1991, pp. 292-296.

It is known to apply hyaluronic acid to burns, open sores, incisions and wounds with the intention of producing adult healing conditions akin to foetal healing conditions. However, these treatments have not been as beneficial as had been hoped.

A way of ameliorating this problem has been sought.

According to the invention there is provided a phospholipid for use in the treatment of a wound by induction of hyaluronic acid secretion.

According to the invention there is further provided a pharmaceutical composition for use in the treatment of a wound by induction of hyaluronic acid secretion which composition comprises a phospholipid in association with a pharmaceutically acceptable diluent or carrier.

According to the invention there is also provided use of a phospholipid or of a pharmaceutical composition according to the invention in the manufacture of a medicament for use in the treatment of a wound by induction of hyaluronic acid secretion.

It has surprisingly been found that when a phospholipid is applied to a wound, it stimulates the production of hyaluronic acid. Without wishing to be bound to any particular theory, it is believed that the invention is beneficial because the phospholipid induces the secretion of autologous hyaluronic acid at the wound site. This is better than externally applied hyaluronic acid which is not necessarily beneficial to the patient because there are many different types of hyaluronic acid each possessing contrasting properties. In the present invention, a phospholipid is used to generate a hyaluronic acid at the wound site, the nature of which is expected to be particular to each patient.

The phospholipid has a phosphatidyl group substituted by an acyl group. The acyl group may comprise a saturated or unsaturated acyl radical generally having from 14 to 22 carbon atoms, preferably from 16 to 20 carbon atoms. Preferably the phospholipid may comprise by way of acyl radicals, the saturated radicals palmitoyl C16:0 and stearoyl C18:0 and/or the unsaturated radicals oleoyls C18:1 and C18:2. Diacyl substitution is preferred and the phospholipid more particularly comprises two identical saturated or unsaturated acyl radicals, especially dipalmitoyl and distearoyl, or a mixture of phospholipids in which such radicals predominate, in particular mixtures in which dipalmitoyl is the major diacyl component.

The phospholipid is optionally either animal-derived or plant-derived or synthetically produced. An artificial phospholipid is generally understood to be a phospholipid that does not occur in nature; preferably it is a synthetic phospholipid free from risk of including animal-derived protein.

The phospholipid is preferably used for the treatment of a wound as the sole active ingredient. Accordingly, the pharmaceutical composition according to the invention preferably only comprises the phospholipid as a therapeutic agent. The phospholipid is preferably substantially free from cholesterol or a tri-glyceride.

A animal-derived phospholipid may be obtained in the usual way by mincing of or lavage from mammalian lungs, such as porcine or bovine lungs. Examples of animal-derived phospholipids which might be used include Curosurf (Chiesi Farmaceutici) which is produced from minced pig lungs and consists of 99% phospholipids and 1% surfactant proteins; Alveofact (Dr. Karl Thomae, Ltd., Germany) which is a compound obtained from bovine lung lavage and contains 90% phospholipids, about 1% proteins, 3% cholesterol, 0.5% free fatty acids, and other components, including triglycerides; Survanta (Abbott, Ltd., Germany) which is prepared by lipid extraction of minced bovine lungs and contains approximately 84% phospholipids, 1% proteins, and 6% free fatty acids; BLES (BLES Biochemicals, Canada) which is produced by a bovine lung lavage; or Infasurf (Forest Labs) also known as calfactant which is produced by bovine calf lung lavage and contains 35 mg/ml phospholipids which are 26 mg/ml phosphatidyl choline (PC), 26 mg/ml dipalmitoylphosphatidylcholine (DPPC), 0.65 mg/ml protein and 0.26 mg/ml a hydrophobic peptide.

A synthetic phospholipid is preferably a diacyl phosphatidyl choline (DAPC) such as DPPC, dioleyl phosphatidyl choline (DOPC) or distearyl phosphatidyl choline (DSPC), phosphatidylglycerol (PG), PC, phosphatidylethanolamine (PE), phosphatidylserine (PS), phosphatidylinositol (PI), phosphatidic acid, and/or a lysophospholipid.

The phospholipid is preferably a mixture of a diacyl phosphatidyl choline and a phosphatidyl glycerol. The phosphatidyl glycerol is advantageously a diacyl phosphatidyl glycerol. The acyl groups of the phosphatidyl glycerol, which may be the same or different, are advantageously each fatty acid acyl groups which may have from 14 to 22 carbon atoms. In practice, the phosphatidyl glycerol component may be a mixture of phosphatidyl glycerols containing different acyl groups. It is preferred for at least a proportion of the fatty acid acyl groups of the phosphatidyl glycerol to be unsaturated fatty acid residues, for example, mono-or di-unsaturated C18 or C20 fatty acid residues.

Preferred acyl substituents in the phosphatidyl glycerol component are palmitoyl, oleoyl, linoleoyl, linolenoyl and arachidonoyl. The phospholipid preferably comprises dipalmitoyl phosphatidyl choline and phosphatidyl glycerol.

The phospholipid is preferably a mixture of DPPC and PG at a weight ratio of from 1:9 to 9:1, preferably from 6:4 to 8:2, more preferably about 7:3. DPPC can be prepared synthetically by acylation of glyceryl phosphoryl choline using the method of Baer & Bachrea, Can. J. of Biochem. Physiol 1959,37, page 953 and is available commercially from Sigma (London) Ltd. PG may be prepared from egg phosphatidylcholine by the methods of Comfurions et al, Biochem. Biophys Acta 1977,488, pages 36 to 42; and Dawson, Biochem J. 1967,102, pages 205 to 210, or from other phosphatidyl cholines, such as soy lecithin.

When co-precipitated with DPPC from a common solvent such as chloroform, PG forms with DPPC a fine powder. Preferably the phospholipid is a mixture of DPPC and a phosphatidyl glycerol derived from egg phosphatidyl choline, which results in phosphatidyl compounds substituted by a mixture of C16, C18 (saturated and unsaturated) and C20 (unsaturated) acyl groups.

Examples of commercial synthetic phospholipid products include: Surfaxin (Discovery Labs) which is also known as lucinactant contains 26 molar parts of DPPC, 8 molar parts of POPG, 5 molar parts of PA and 1 part of KL-4; Lung Surfactant Factor LSF (Altana) which is also known as lusupultide contains recombinant SP-C, DPPC, PG and PA; Exosurf (GSK, Germany) which is composed of DPPC (˜84%), cetyl alcohol, and tyloxapol; or pumactant (Britannia Pharmaceuticals) which is composed of a mixture of DPPC and PG at a weight ratio of 7:3, may be used in the invention.

The phospholipid is preferably a phospholipid or a mixture of phospholipids which has a melting temperature which is about the same as or below body temperature (which is the temperature of the human or animal body to be treated). Such a mixture of phospholipids preferably contains a spreading phospholipid which has a melting temperature which is about the same as or below body temperature such as PG, PE, PS, or PI.

The phospholipid is preferably applied at a rate of from 1, preferably from 10, more preferably from 50 to 1000, preferably to 800, more preferably to 300 μg per square centimetre of wound.

The phospholipid is preferably applied in the form of a dry powder. More generally, the powdered phospholipid may have a particle size in the range of 0.5 to 100 μm, more suitably of 0.5 to 20 μm, preferably 0.5 to 10 μm. The phospholipid is preferably a surface active phospholipid (SAPL).

The phospholipid or composition according to the invention preferably induces hyaluronic acid secretion in an epithelial or mesothelial cell.

The wound to be treated by the invention is preferably an opening or abrasion on a surface of a human or animal body. The surface of a human or animal body to be treated is optionally either an internal or external surface. The wound is preferably physical trauma. It may be internal or external physical trauma, for example hurt or injury caused by a burn, accidental or non-surgical incision, violent or disruptive action. The internal physical trauma treated by the invention is internal accidental physical trauma caused by injury as a result of an accident or unintentional action. External physical trauma to be treated by the present invention includes external accidental physical trauma as well as external surgical physical trauma caused by surgery such as for example trauma caused by the removal of skin for a skin graft. The wound is optionally a site on the surface of a human or animal body where the condition of the surface is accidentally degraded such as a burn, opening or abrasion.

The treatment of a wound in the invention preferably comprises facilitating re-epithelialisation, particularly of a keratinocyte cell. In other words, the phospholipid is preferably used to treat a wound by facilitating re-epithelialisation. By re-epithelialisation is meant re-growth of epithelial or other surface cells. Furthermore the treatment of a wound in the invention preferably promotes wound healing by wound closure. The phospholipid or pharmaceutical composition according to the invention is preferably applied topically to the wound.

The pharmaceutical composition according to the invention comprises a pharmaceutically acceptable excipient. Any compatible excipient may be used. The excipient is preferably free from water. Where the carrier or diluent is a liquid, it is preferably non-aqueous. The excipient preferably comprises a surface active agent. A surface active agent is useful because it enables a phospholipid having a melting temperature above body temperature to be used in the composition. More preferably the surface active agent is a pharmaceutically acceptable surfactant or hydrophobic protein. Examples of such agents include: KL-4 which is 21 amino acid synthetic peptide; tyloxapol which is a nonionic surfactant; cetyl alcohol (or hexadecanol); or cholesteryl palmitate. A further suitable excipient is a protein, especially a protein which improves absorption such as apoprotein B.

When the composition is provided in liquid form, the excipient preferably comprises a carrier liquid in which the phospholipid is dispersed or dissolved. The carrier liquid is typically one which is substantially non-volatile or only sparingly volatile at body temperature. A suitable carrier includes a physiologically acceptable glycol, especially a propylene glycol, polyethylene glycol and/or glycerol.

The composition may optionally be provided in liquid, semi-liquid or pasty form. Pastes can be prepared by simply dispersing a phospholipid in a suitable carrier, or, when appropriate, dissolving the phospholipid in a heated carrier and allowing the phospholipid to precipitate as a powder on cooling, preferably at a loading that will form a paste. Propylene glycol is especially effective as a carrier because at room temperature a phospholipid may be dispersed in it as a paste, but at body temperature a mobile solution is formed. Also polyethylene glycols may be prepared which are waxy solids at room temperature and liquids at body temperature, such as for example PEG 600. Various dispersions of a phospholipid in propylene glycol are described in U.S. Pat. No. 6,133,249, the entire contents of which are incorporated herein by reference.

According to the invention, there is further provided a method of treating a wound which method comprises applying to a human or animal patient in need of such treatment a therapeutically effective amount of a phospholipid. The phospholipid is preferably in the form of a pharmaceutical composition according to the invention.

The invention is illustrated with reference to the following Figures of drawings which are not intended to limit the scope of the claims and in which:

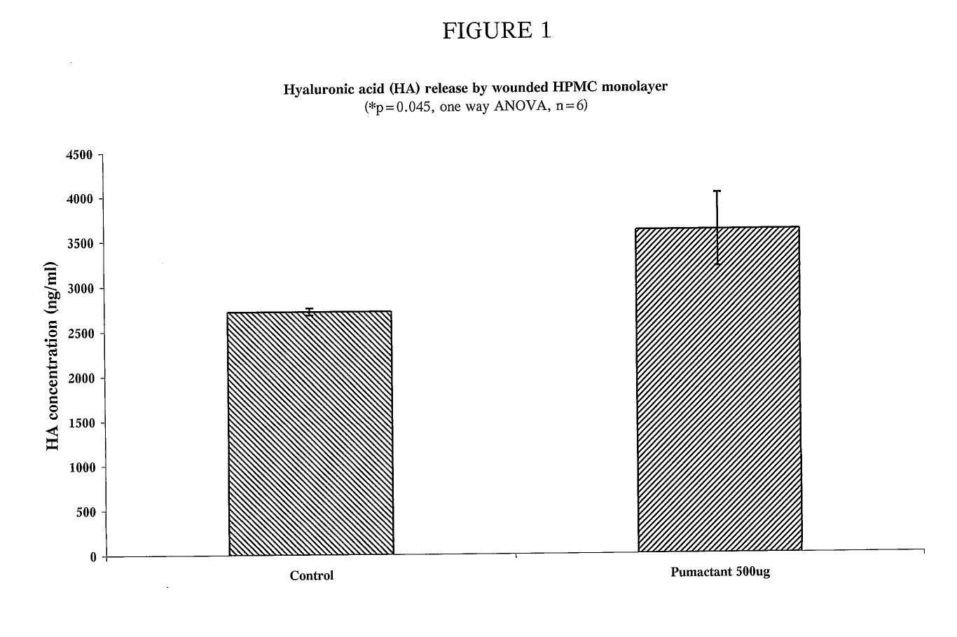

FIG. 1 is a graph comparing hyaluronic acid release by a monolayer of wounded human peritoneal mesothelial cells treated by 500 μg of pumactant (referred to as Adsurf) with control;

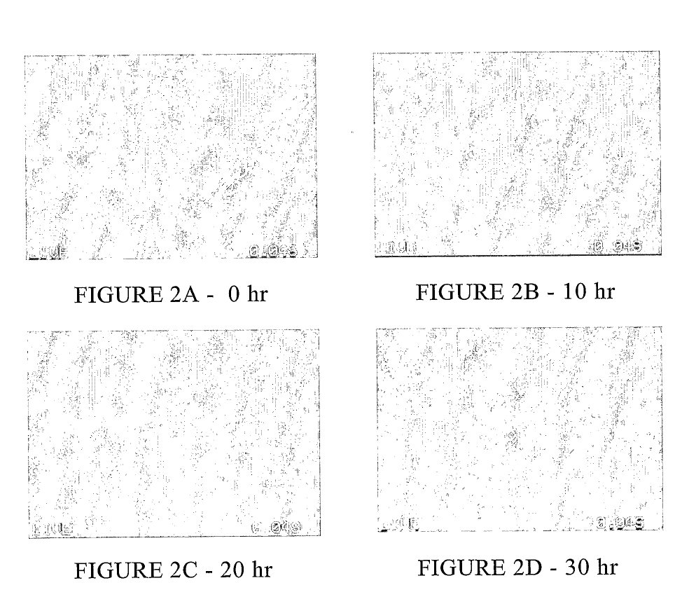

FIG. 2 is a series of images of re-mesothelisation of control cells taken using a digital video camera where FIG. 2A was taken at 0 hours, FIG. 2B was taken at 10 hours, FIG. 2C was taken at 20 hours and FIG. 2D was taken at 30 hours;

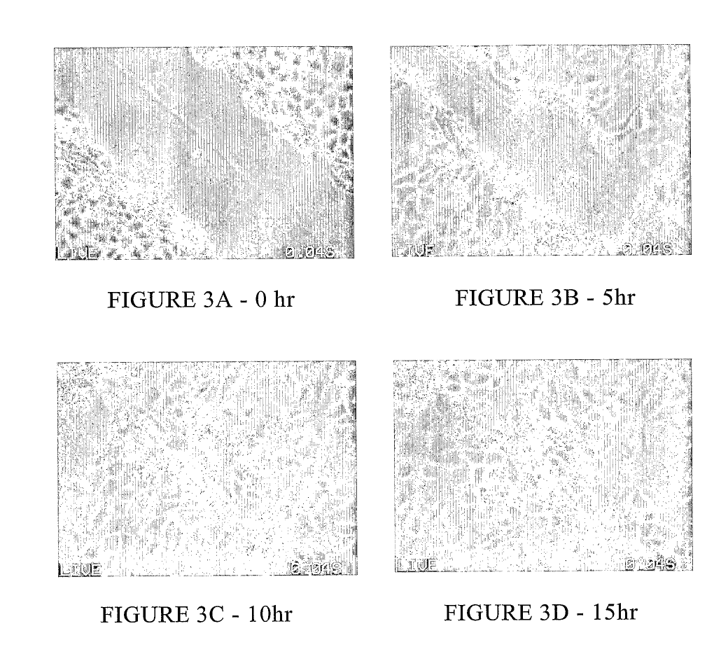

FIG. 3 is a series of images of re-mesothelisation of cells treated with pumactant taken using a digital video camera where FIG. 3A was taken at 0 hours, FIG. 3B was taken at 5 hours, FIG. 3C was taken at 10 hours and FIG. 3D was taken at 15 hours;

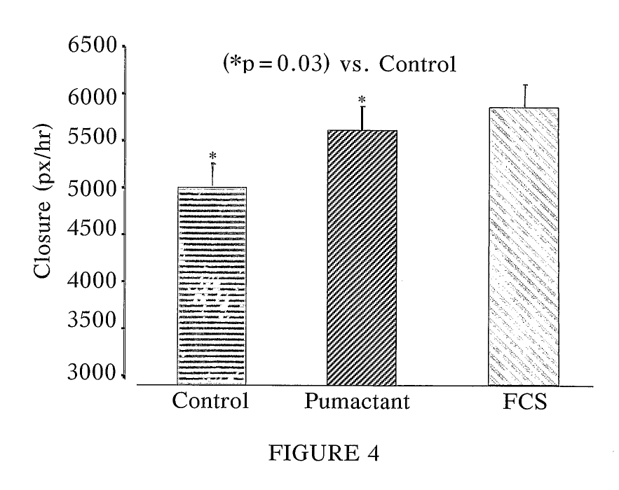

FIG. 4 is a graph comparing the rate of closure of a wound for cells treated by pumactant or foetal calf serum with a control; the graph has a scale on its y-axis of pixels per hour obtained from timed images from a digital camera;

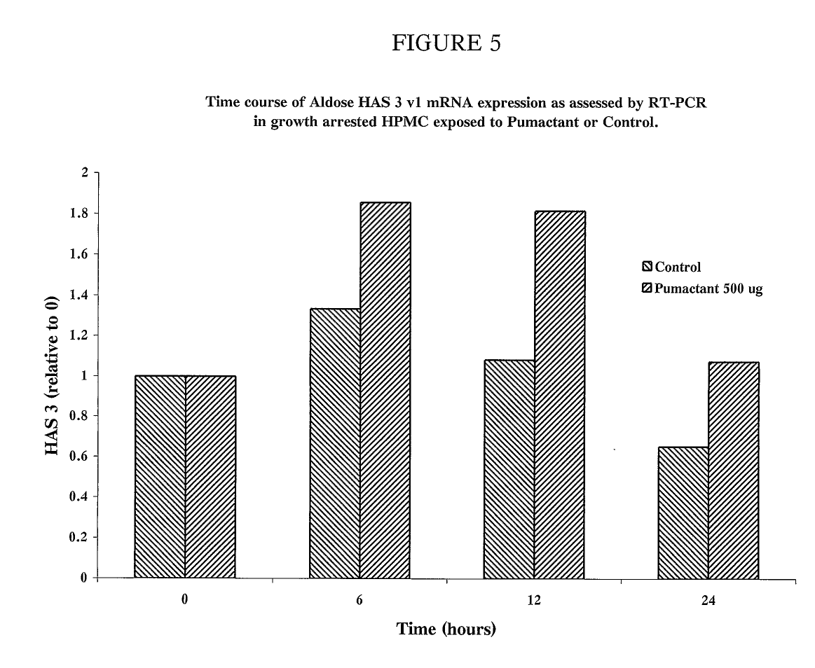

FIG. 5 is a graph showing the time course of aldose HAS3 v1 mRNA expression as assessed by RT-PCR in growth arrested HPMC exposed to pumactant or control; bars represent the mean HAS 3 v1/control (adjusted to 1) expression derived from densitometric scanning of the negative images of the gels obtained; and

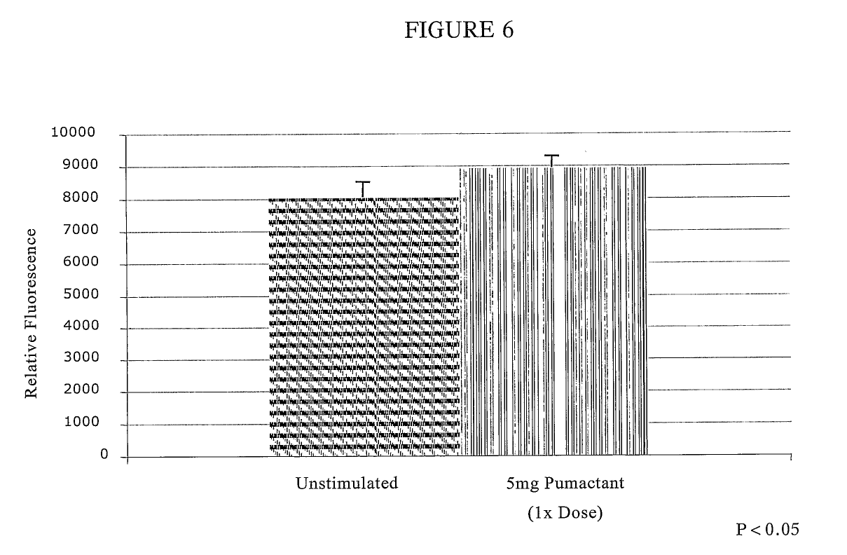

FIG. 6 is a graph showing the relative fluorescence as measured by the Alamar Blue™ (Biosource) assay which is a measurement of the proliferation of unstimulated cells or cells treated by pumactant.

The invention will now be illustrated with reference to the following Examples which are not intended to limit the scope of the claims.

### Example 1

Growth arrested HPMC cultured on 6 well plates were scratch wounded in a linear fashion and were followed by microscopy until wound healing was observed (96 hours). Half of the wells were treated with 500 micrograms of dry pumactant per square centimetre, whilst half were left in serum free conditions (control). Following the 96 hour period, supernatants from each well were taken, centrifuged and stored at −70° C. until analysis. Supernatants were assayed for hyaluronic acid using a commercially available ELISA kit (Corgenix Inc.) according to the manufacturer's instructions. The mean hyaluronic acid content was determined and compared with control as shown in FIG. 1.

It can be seen that surprisingly pumactant induces the secretion of hyaluronic acid in mesothelial cells.

### Example 2

In this Example, the effect of pumactant on promoting remesothelialisation was investigated using a validated mesothelial wound healing model (Yung S, Davies M. Response of the human peritoneal mesothelial cell to injury: an in vitro model of peritoneal wound healing. Kidney Int. 1998 December; 54(6):2160-9).

HPMC (human peritoneal mesothelial cells which are primary cells cultured from individual patients undergoing abdominal surgery) were seeded onto 24-well culture plates. Following growth arrest in serum-free medium the monolayer was injured by mechanical linear scraping with a sterile pipette tip to leave a reproducible area devoid of cells or extra-cellular matrix. The well was washed with serum free medium to remove detached cells. Pumactant (500 μg) was applied in a dry form per square centimetre of the monolayer using a pulse applicator (SMC Pneumatics, Milton Keynes, UK), and serum free medium (1 mL) replaced. The controls used were serum free medium alone, or medium supplemented with 2% (v/v) FCS. The denuded area in each well was identified microscopically and the coordinates recorded for subsequent data capture.

Re-mesothelialization was continuously monitored using an Axiovert 100M inverted microscope fitted with a computer-controlled XY automated scanning stage and incubator. The humidified incubator was maintained at 37° C. and 5% CO₂ with a heated insert and vectorial airflow (Carl Zeiss, Oberkochen, Germany). Images were captured from the same position in each well of the 24-well plates, using the 2.5× objective, at 60-minute intervals on an Orca C5985 digital video camera (Hamamatsu Photonics, Hamamatsu City, Japan). Images were analyzed using Openlab version 3.0.8 on a Macintosh G4 computer (Improvision, Ltd., Coventry, UK). The rate of re-mesothelialization was calculated by measuring the reduction of the denuded area (in pixels) at 60-minute intervals until wound closure was seen.

Mesothelial wound healing was complete by 20 hours in serum free control conditions (as can be seen from FIG. 2). With pumactant applied as a dry powder, mesothelial healing was enhanced above control (as can be seen from FIGS. 3 and 4). Therefore it can be seen that pumactant positively encourages wound closure.

### Example 3

In this Example the effect of pumactant on the up-regulation of hyaluronan synthase (HAS) genes was investigated.

Monolayers of HPMC cultured in M199 medium supplemented with 10% FCS on 6 well plates, were growth arrested and either scratch wounded in a cross-shaped fashion or left untouched and then the wells washed with serum free medium to remove detached cells. Both wounded and unwounded cells were divided into two treatments groups where HPMC were either treated with dry pumactant (500 μg) per square centimetre of the monolayer using a pulse applicator and serum free medium or treated with serum free medium alone. The cultures were then observed over a 96 hour time period. Over the 96 hour time course supernatants from duplicate cultures were removed at the following time points; 0, 6, 12, 24, 48 and 96 hours and stored at −20° C. until required.

At each time point total RNA was extracted from HPMC by lysing the cell membranes to release nuclear contents using TRI Reagent (Sigma, Poole, UK) and then cDNA was reverse transcribed using Superscript II RNase H⁻ reverse transcriptase (InVitrogen, Paisley, Scotland) and random hexamers (100 μm) (Amersham, Bucks, UK) from 1 μg of total RNA.

HAS gene specific (HAS I, HAS II and HAS III v.1) PCR reactions were performed using 2 μl of the cDNA products obtained at each time point in a total volume of 50 μl with a 1× PCR buffer (1.5 mM Mg Cl₂, 0.2 mM dNTPs, 2.5 Units of Amplitaq Gold Taq polymerase and 1 μM of HAS specific oligonucleotide PCR primers (see below).

HAS gene specific Primer Sequences:

| HAS I | |
|---|---|
| (SEQ ID No. 1) | |
| Forward | ACT GGG TAG CCT TCA ATG TGG A |
| (NM001523) | |
| (SEQ ID No. 2) | |
| Reverse | GAC GAG GGC GTC TCT GAG TAG |
| HAS II | |
| (SEQ ID No. 3) | |
| Forward | CAT AAA GAA AGC TCG CAA CAC G |
| (NM005328) | |
| (SEQ ID No. 4) | |
| Reverse | ACT GCT GAG GAA TGA GAT CCA G |
| HAS III v.1 | |
| (SEQ ID No. 5) | |
| Forward | CAG CAC CTT CTC GTG CAT CA |
| (NM005329) | |
| (SEQ ID No. 6) | |
| Reverse | ACT GCA CAC AGC CAA AGT AGG A |

The cDNA was then denatured at 94° C. for 2 minutes and amplified with 38 cycles (30 seconds at 94° C., 30 seconds at 65° C. and 90 seconds at 68° C.) concluding with an extension step of 15 minutes at 68° C.

Reaction products from each time point and each treatment were analysed by separation on a 15% electrophoretic agarose gel, the gel visualised and photographed using the BioRaD image system and QuantityOne software. Densitometric scanning of the negative images of the gels provided the normalised quantitative data of mRNA expression of the specific HAS genes shown in FIG. 5.

It can be seen that pumactant induces the up-regulation of HAS gene expression in HPMC in a time dependent manner.

### Example 4

The effect of the pumactant on keratinocyte proliferation was examined using the AlamarBlue™ Assay (Biosource). Briefly, this assay is designed to measure the proliferation of various cell types by measuring the ability of the cells to metabolise and reduce a REDOX indicator (Alamar blue). Reduction of the REDOX indicator results in a change in its fluorescent activity. Therefore the fluorescent activity measured is proportional to cell proliferation.

The cells used were Human Epidermal Keratinocytes (Adult). They were purchased from TCS Cellworks Ltd (Product code ZHC-1111).

| | | |
|---|---|---|
| BATCH NUMBER: | 26619T | |
| DONOR INFORMATION: | Age: | 39 years |
| | Sex: | female |
| | Pigmentation: | Dark |

### Proliferation Assay

The effect of the pumactant on keratinocyte proliferation was examined using the AlamarBlue™ Assay (Biosource). Briefly, this assay is designed to measure the proliferation of various cell types by measuring the ability of the cells to metabolise and reduce a REDOX indicator (Alamar blue). Reduction of the REDOX indicator results in a change in its fluorescent activity. Therefore the fluorescent activity measured is proportional to cell proliferation.

The assay involves the addition of 10% Alamar blue to the cells and a subsequent 1 hour incubation. After 1 hour, cell proliferation was quantified by measuring the fluorescent activity of Alamar blue at 540 nm (wavelength).

### Experiment Methodology

Approximately 5 mg (single dose) of pumactant was applied to sub-confluent keratinocytes (24 well plate, passage 2). Cells with no pumactant stimulation were used as a control. The proliferation assay was carried out on both stimulated and un-stimulated cells. Fluorescent activity is shown in FIG. 6 as “Relative Fluorescence” or “Arbitrary Units”.

### Result

The pumactant-stimulated cells appear to proliferate more than the un-stimulated control cells (p<0.05) suggesting the pumactant can induce an increase (approximately 10%) in proliferation of human keratinocytes. These initial data is consistent with the data in Example 2 regarding human peritoneal mesothelial cell proliferation.

### Notes

This experiment was carried out on cells that were sub-confluent (few cells) and at an early time point (12 hours) and the data should be regarded as preliminary data.

1-23. (canceled)

24. A method of treating a wound which method comprises applying to a human or animal patient in need of such treatment a therapeutically effective amount of a phospholipid to induce hyaluronic acid secretion wherein the wound is an external physical trauma or an internal physical trauma caused by injury as a result of an accident or unintentional action.

25-26. (canceled)

27. A method as defined in claim 24 wherein the wound is an opening or abrasion on an internal or external surface of a human or animal body

28. A method as defined in claim 27 wherein the animal body is a mammalian body.

29. A method as defined in claim 24 wherein the wound is physical trauma.

30. A method as defined in claim 24 wherein the phospholipid facilitates re-mesothelialisation.

31. A method as defined in claim 24 wherein the phospholipid is a synthetic phospholipid

32. A method as defined in claim 24 wherein the phospholipid is a diacyl phosphatidyl choline, phosphatidyl glycerol, phosphatidyl choline, phosphatidyl ethanolamine, phosphatidyl serine, phosphatidyl inositol, phosphatidyl glycerol, phosphatidic acid, and/or a lysophospholipid.

33. A method as defined in claim 24 wherein the phospholipid is a mixture of dipalmitoylphosphatidylcholine and phosphatidylglycerol at a weight ratio of from 1:9 to 9:1.

34. A method as defined in claim 24 wherein the phospholipid is used at a rate of from 1, preferably from 10, more preferably from 50 to 1000, preferably to 800, more preferably to 300 μg per square centimetre of wound.

35. A method as defined in claim 24 wherein the phospholipid is in the form of a dry powder.

36. A method as defined in claim 24 wherein the phospholipid is the only active ingredient for use in the treatment of a wound.

37. A method as defined in claim 24 wherein the treatment of a wound comprises facilitating re-epithelialisation.

38. A method as defined in claim 24 wherein the phospholipid is in the form of a pharmaceutical composition which comprises a phospholipid in association with a pharmaceutically acceptable excipient.

39. A method as defined in claim 38 wherein the excipient is a surface active agent, a protein, and/or a carrier liquid.

40. A method as defined in claim 29 wherein the physical trauma is accidental physical trauma or external surgical physical trauma.

41. A method as defined in claim 33 wherei the phosophlipid is a mixture of dipalmitoylphosphatidylcholine and phosphatidyglycerol at a weight ratio of from 6:4 to 8:2.

42. A method as defined in claim 33 wherein the phospholipd is a mixture of dipalmitoylphosphatidylcholine and phosphatidylglycerol at a weight ratio of about 7:3.
